(19) United States
(12) Patent Application Publication
(10) Pub. No.: US 2017/0348136 A1
(43) Pub. Date: Dec. 7, 2017

(54) A DEVICE FOR ENHANCING AND/OR MAINTAINING A PENILE ERECTION

(71) Applicant: Exia Biomedical Inc., Ottawa (CA)

(72) Inventor: Nigel Andrew Shaw, Ottawa (CA)

(21) Appl. No.: 15/122,591

(22) PCT Filed: Feb. 22, 2016
(86) PCT No.: PCT/CA2016/050173

§ 371 Date: Aug. 30, 2016

Related U.S. Application Data
(60) Provisional application No. 62/119,585, filed on Feb. 23, 2015.

(51) Int. Cl. A61F 5/41

(52) U.S. Cl. CPC A61F 5/41; A61F 2005/414

(57) ABSTRACT

The present invention relates to a device for treatment of erectile dysfunction comprising a ring. The device further provides for retraction of the foreskin of the user and maintains the retraction of the foreskin throughout sexual activity. Such a device also increases the length of the sliding interface of the penis during sexual activity. Further, the device will stimulate the stretch-sensitive nerve endings of the foreskin before and during sexual activity. Further, the device will increase stimulation of the sensitive nerve endings of the glans during sexual activity. The device will also retain blood within the penis upon erection, allowing for more reliable, harder and longer erection periods. The device also creates the appearance of a larger penis.

### FIELD OF THE INVENTION

The present invention pertains to the field of male sexual health. In particular, the present invention provides a device for enhancing and/or maintaining a penile erection and optionally providing foreskin retraction or restraint.

### BACKGROUND

Erectile dysfunction (ED) is a prevalent condition that affects the physical and psychosocial well-being and quality of life for millions of men, their partners, and families. Achieving and maintaining an erection requires, in the most basic sense, sufficient blood flow to the penis and sufficient restriction of blood flow out of the penis. ED occurs when the blood flow in is too little, the blood flow out is too great, or a combination of the two.

The Canadian Study of Erectile Dysfunction identified 49.4% of men over 40 with ED, with other studies showing that 5% to 20% of men have moderate to severe ED. First line treatment for ED include phosphodiesterase type 5-(PDE5) inhibitors such as sildenafil (Viagra), tadalafil (Cialis) and vardenafil (Levitra). Use of PDE5 inhibitors is contraindicated for men taking nitrate medications. In addition, a number of adverse drug reactions and drug interactions have been identified and as a result prevent the use of PDE5 inhibitors for the treatment of ED in some men. Moreover the high cost of PDE5 inhibitors may further limit the availability of this treatment option for some affected men.

Currently available second-line treatment options include self-injection with vasoactive agents, vacuum erection devices, and surgical approaches with inflatable penile prostheses. Other second-line treatment options include restricting blood flow out of the penis. The common penis ring or “cock ring” works in principle by this mechanism, however longitudinal movement of the ring along the shaft of the penis allows blood to escape thereby reducing its overall effectiveness as a treatment for ED. To improve functionality of the penis ring, the penis ring may be made very tight. This however leads to discomfort and increased risk of injury. Variations of the penis ring are known in the art and include penis erection stabilizer disclosed in U.S. Pat. No. 6,319,194. Other erection aid devices include the simplified erection aid device disclosed in U.S. Pat. No. 5,728,043.

Both physical stimulation and erotic or emotional stimulation trigger and help maintain an erection. In non-circumcised or partially circumcised males, the foreskin slides, or “rolls” back and forth over the shaft of the erect penis during sexual stimulation to a greater or lesser extent depending on the amount of foreskin present. While the sliding and rolling of the foreskin has in and of itself a certain stimulating effect, it also has the potential to reduce overall penile stimulation. The foreskin alternately covers and uncovers the head, or glans, of the penis, and in some cases does not completely uncover the glans, thereby reducing the direct frictional stimulation of the glans, which is the most sensitive part of the penis, and; it reduces the effective length of the sliding interface between the shaft of the penis and the inner wall of the vagina or alternate stimulating object or body part, on any insertion or retraction stroke by an amount equal to the length of the sliding of the foreskin. Devices for restraining penile foreskin are known in the art and include the device disclosed in U.S. patent application Ser. No. 10/325,598 (published as US 2011/0146695).

This background information is provided to reveal information believed by the applicant to be of possible relevance to the present invention. No admission is necessarily intended, nor should be construed, that any of the preceding information constitutes prior art against the present invention.

### BRIEF SUMMARY

An object of the present disclosure is to provide a device for enhancing and/or maintaining a penile erection. In accordance with an aspect of the invention, there is provided a device comprising a stretchable substantially oval-shaped or substantially circular first band having a proximal edge and distal edge and sized to restrict blood flow out of a user's penis without substantially impending blood flow into the penis; and a stretchable second band having a first end and a second end, wherein the first end and the second end are attached to the proximal edge of the first band at substantially diametrically opposed points, wherein the second band is sized to fit around a user's waist and upon use the device is worn around the user's waist and the first band is pulled forward between the user's legs and stretched over the penis and positioned at the base of the penis such that the proximal edge is towards the user and the second band is under tension thereby exerting a pull on the first band towards the base of the penis.

In accordance with another aspect of the present invention, there is provided a kit comprising the device described above and instructions for use and optionally one or more condoms.

In accordance with another aspect of the present invention, there is provided a method of treating ED wearing the device described above.

In accordance with another aspect of the present invention, there is provided use of the device described above to treat ED.

### BRIEF DESCRIPTION OF THE FIGURES

Embodiments of the present invention will now be described, by way of example, by reference to the attached Figures, wherein:

FIG. 1A illustrates a perspective view of the device (1) in accordance with at least one embodiment of the present invention detailing the first band (i.e. “ring”) (10) and second band (i.e. “loop”)(20).

FIG. 1B illustrates part of a device in accordance with at least one embodiment of the present invention detailing sizing bands (30).

FIG. 2A illustrates lubrication of the device prior to use.

FIG. 2B illustrates a posterior view of a user wearing the device of FIG. 1 prior to insertion of the penis into the ring. The device is initially worn such that the ring is positioned in-line with the intergluteal cleft and the loop sits against the abdominal wall below the umbilicus but generally above the anterior superior iliac spines.

FIG. 2C illustrates a posterior view of a user wearing the device of FIG. 1 reaching between his legs to grasp the ring.

FIG. 2D illustrates an anterior view of the user of FIG. 2C having pulled the ring to the front and in the process of slipping the ring over the head of the penis.

FIG. 2E illustrates an anterior view of the user of FIGS. 2C and 2D detailing final placement of the device at the base of the penis. The second band (20) is also shown and one of its attachment points (25) on the ring (10).

FIG. 2F illustrates an anterior view of the user of FIGS. 2C, 2D and 2E detailing placement of the ring relative to the erect penis and scrotum. The figure shows the second band pulling the ring posteriorly.

FIG. 2G illustrates an anterior view of the user of FIG. 2F detailing the stretch-sensitive nerve effect.

FIG. 2H illustrates an anterior view of the user of FIG. 2G showing swelling of the penis surface flesh and glans due to engorgement.

FIG. 3 illustrates user of FIG. 2 engaging in vaginal intercourse detailing part of the ring (10) and second bands (20) and its attachment site.

FIG. 4A is a closeup of the user and device shown in FIG. 2F with left leg abducted and drawn back.

FIG. 4B is a side view of the user and device shown in FIG. 2F showing that the scrotum is pulled back by the ring until it is substantially posterior of the plane in-line with the anterior superior iliac spines and pubic symphysis.

### DETAILED DESCRIPTION

This invention provides a device for enhancing and/or maintaining a penile erection and methods of using the device to treat ED. For users with a fully or partially intact foreskin, the device further provides for foreskin retraction and/or restraint. The device is configured to be worn during sexual activity including intercourse or alternate penile stimulation and is designed to enhance the sexual experience.

The device is configured to slow blood flow from the penis, stimulate the stretch-sensitive nerve endings of the penile skin, promote engorgement of the penis, prevent penile skin from sliding back and forth during intercourse thereby (a) increasing stimulation of the glans and (b) increasing the effective frictional length, draw the testicles and scrotum posteriorly, and prevent testicle “ride-up” during intercourse.

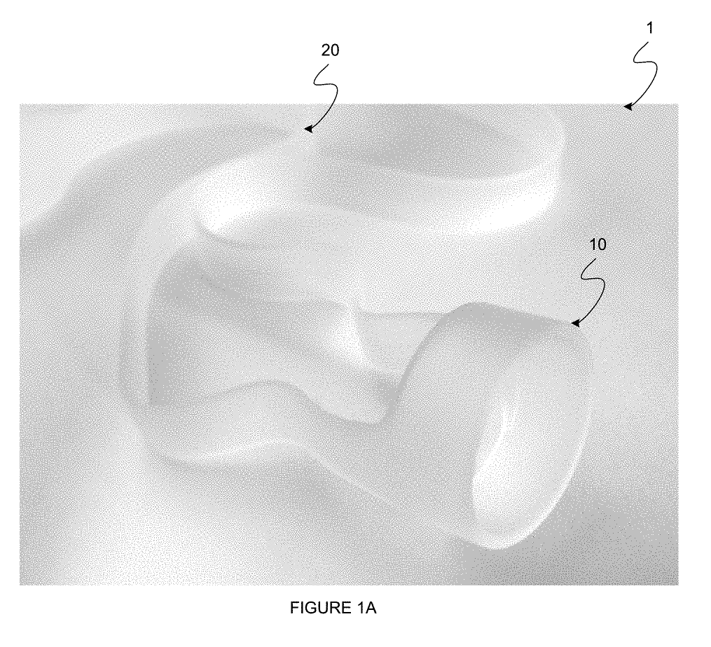

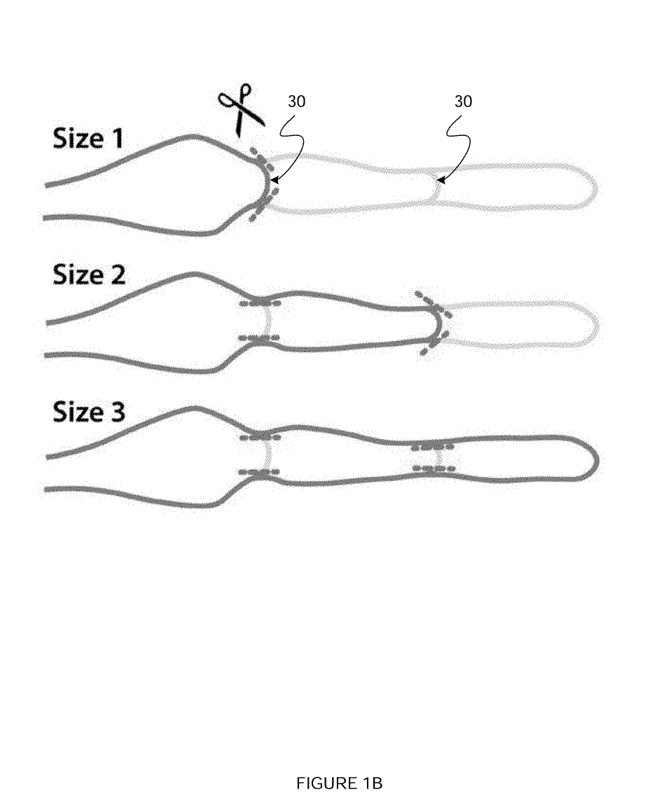

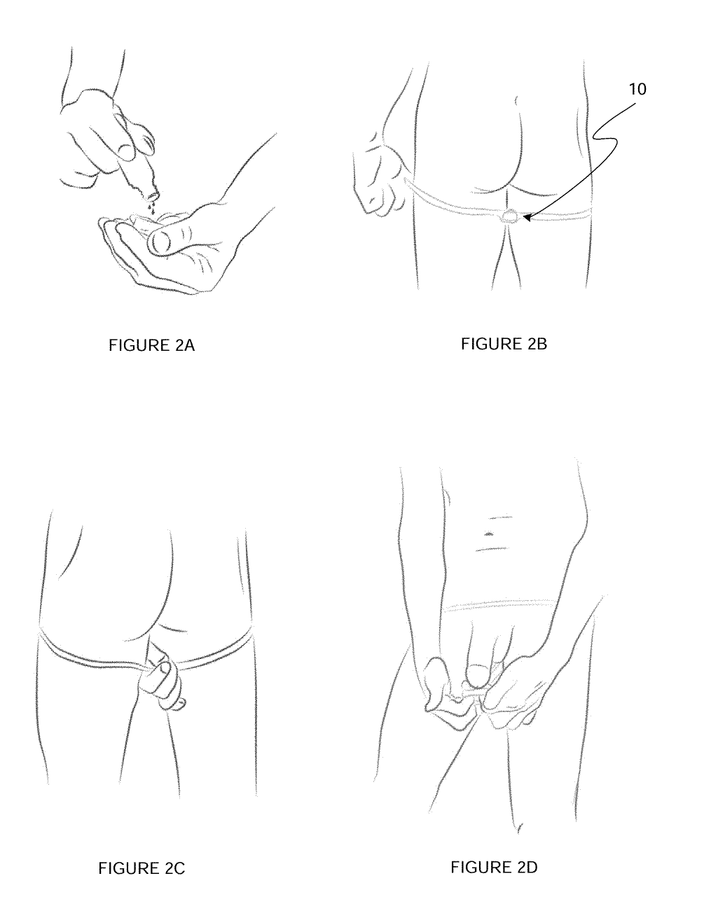

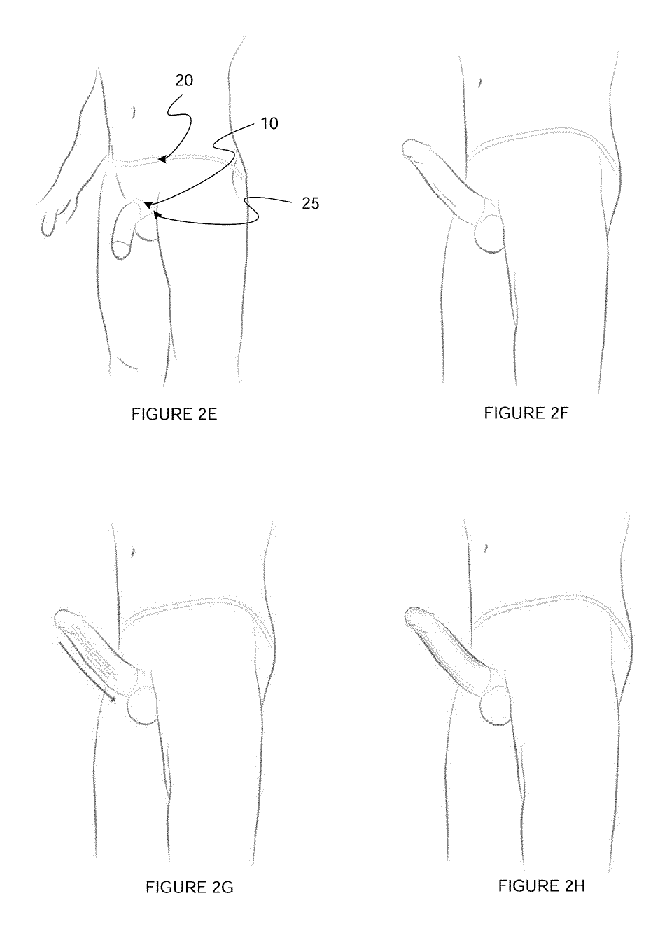

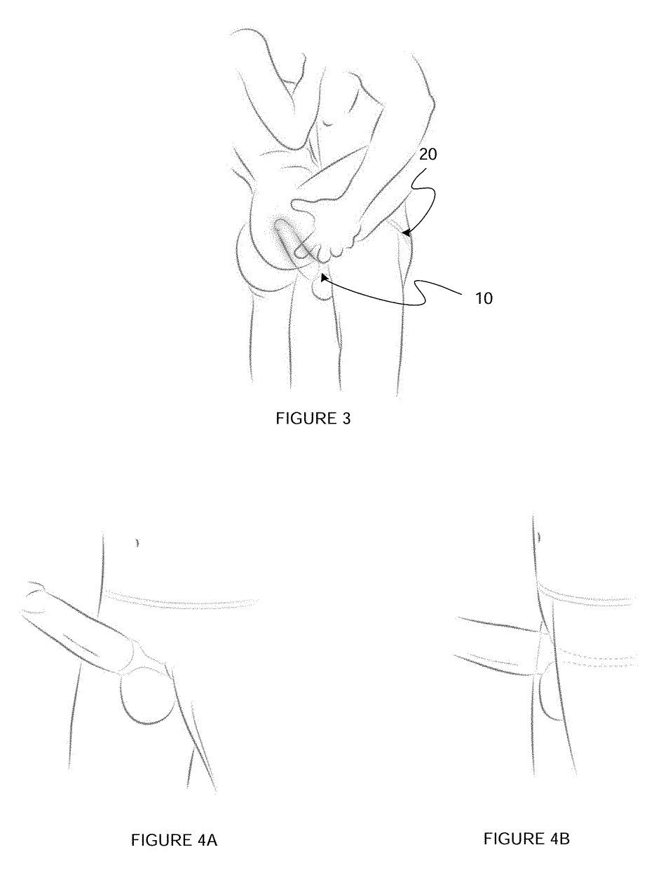

Referring to FIG. 1, the device comprises a stretchable, optionally elastic or latex, substantially oval-shaped or substantially circular band, i.e. “ring” sized to restrict blood flow out of the penis without substantially impending blood flow into the penis. The ring is attached to or is integral with the ends of a stretchable, optionally elastic or latex, second band at its proximal edge at substantially diametrically opposed points such that the second band forms a loop extending from the ring and is referred to as the “loop” herein. The loop is of sufficient size that when worn around the user's waist with the ring at the back, the ring may be pulled forward between the user's legs and stretched over the penis at its base. During use, the second band is under tension and thereby exerts a pull on the ring in the direction of the base of the penis.

In some embodiments, the loop may include one or more sizing bands as shown in FIG. 1B, such that the size of the loop may be adjusted by cutting one or more of these bands. In addition or alternatively, the device may be manufactured in a variety of sizes.

The device may be made of any other appropriate material contemplated by someone skilled in the art including but not limited to elastic, latex, or silicone. In some embodiments, the device is made of a single material and the ring and second band optionally integral. Alternatively, the ring and the second band may be made as separate pieces and fused or otherwise attached.

In other embodiments, the ring and the loop are made from different materials.

In some embodiments, the ring is made of silicone in order to reduce the friction of the ring on the penile skin.

The device may be manufactured in any desired colour. In some embodiments, it is contemplated that the device will be clear in appearance in order to blend in with the flesh colour of the user. Alternatively, the device may be solid in colour or patterned or textured in order to accentuate the device during sexual activity. It is also contemplated that the material of the device could have certain appearance and functional properties that enhance sexual activity, such as leather-like, glow in the dark material, and/or other materials understood by a skilled person in the field.

The device, by slowing blood flow from the base of the penis, facilitates the start and maintenance of an erection. The ring of the device exerts a mild compressive force at the base of the penis thereby partially restricting blood flow from the penis resulting in a more reliable, harder and more lasting erection. The device applies pressure to the dorsal veins, both the superficial dorsal and the deep dorsal and also provides supportive pressure on the two corpus cavernosa helping to enable adequate compressive force.

The device, by facilitating the engorgement of the soft surface flesh of the penis, makes the penis look and feel thicker and as a result may have a positive psychological impact on the user.

The device, by holding the ring in place at the base of the penis, allows for the ring to be of a more comfortable and safe size while still being effective for treating erectile dysfunction.

The second band (i.e. loop) is under tension once the user puts on the device. This tension pulls the ring posterior which results in the scrotum being pulled posterior. Since the skin of the scrotum is continuous with the penile skin, the base of the penile skin is also pulled in the direction of the base of the penis thereby stimulating the stretch-sensitive nerves of the penile skin. This may facilitate a stronger erection.

In users with an intact or partially intact foreskin, this penile skin pulling has the effect of retracting the foreskin and ensuring the foreskin remains retracted, facilitating the extension of glans of the penis through the foreskin. The tension provided by the device also acts to retain the foreskin and in particular during prolonged periods of sexual activity. It also prevents the foreskin from sliding back and forth during intercourse, thus preventing the foreskin from covering and uncovering the head of the penis, thereby increasing direct frictional stimulation of the head. When the foreskin is retained, the sensitive nerves of the glans are exposed to stimulation. By prolonging foreskin retraction, the sensitive nerves of the glans remain exposed for the entirety of sexual activity. The user may therefore experience greater stimulation of these sensitive nerves and potentially greater sexual enjoyment. By prolonging foreskin retraction, the effective frictional length of the intercourse stroke is increased because the stroke no longer has to take up the slack in the foreskin. The user and/or user's sexual partner may therefore experience a sense of increased stimulation and greater penile length.

During use, the device pulls the scrotum posterior longitudinally relative to the penile shaft somewhat beyond the location at which it normally rests and maintains the scrotum in that position. The user or an onlooker such as the user's sexual partner may therefore perceive the penis as having greater length.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

The scope of the claims should not be limited by the preferred embodiments set forth in the foregoing, but should be given the broadest interpretation consistent with the description as a whole.

1. A device comprising a stretchable substantially oval-shaped or substantially circular first band having a proximal edge and distal edge and sized to restrict blood flow out of a user's penis without substantially impending blood flow into the penis; and
a stretchable second band having a first end and a second end, wherein the first end and the second end are attached to the proximal edge of the first band at substantially diametrically opposed points, wherein the second band is sized to fit around a user's waist and upon use the device is worn around the user's waist and the first ring is pulled forward between the user's legs and stretched over the penis and positioned at the base of the penis such that the proximal edge is towards the user and the second band is under tension thereby exerting a pull on the first band towards the base of the penis.

2. The device of claim 1, comprising one or more sizing bands.

3. The device of claim 1, wherein the device is a unitary structure.

4. The device of claim 1, wherein the device is made of latex.

5. A kit comprising the device of claim 1 and instructions for use.

6. The kit of claim 5, further comprising lubricant.

7. The kit of claim 5, further comprising one or more condoms.

8. A method of treating erectile dysfunction comprising wearing the device according to claim 1.

9. (canceled)

10. The device of claim 2, wherein the device is a unitary structure.

11. The device of claim 2, wherein the device is made of latex.

12. The device of claim 3, wherein the device is made of latex.

13. A kit comprising the device of claim 2 and instructions for use.

14. A kit comprising the device of claim 3 and instructions for use.

15. A kit comprising the device of claim 4 and instructions for use.

16. The kit of claim 13, further comprising lubricant.

17. The kit of claim 14, further comprising lubricant.

18. The kit of claim 15, further comprising lubricant.

19. A method of treating erectile dysfunction comprising wearing the device according to claim 2.

20. A method of treating erectile dysfunction comprising wearing the device according to claim 3.

21. A method of treating erectile dysfunction comprising wearing the device according to claim 4.
